(19) United States
(12) Patent Application Publication
(10) Pub. No.: US 2017/0107482 A1
(43) Pub. Date: Apr. 20, 2017

(54) ANIMAL CELL REGENERATION SPEED CONTROLLING METHOD AND ANIMAL CELL REGENERATION SPEED CONTROLLER

(71) Applicant: TOYOTA BOSHOKU KABUSHIKI KAISHA, Aichi-ken (JP)

(72) Inventor: Yuko YAMAMOTO, Aichi-ken (JP)

(73) Assignee: TOYOTA BOSHOKU KABUSHIKI KAISHA, Aichi-ken (JP)

(21) Appl. No.: 15/273,126

(22) Filed: Sep. 22, 2016

(30) Foreign Application Priority Data
Oct. 20, 2015 (JP) 2015-206716

(51) Int. Cl. C12N 5/07

(52) U.S. Cl. CPC C12N 5/0601; C12N 2527/00; C12N 2525/00

(57) ABSTRACT

The present invention is a method for controlling a regeneration speed of an animal cell that includes a step of grasping tide-generating force and a step of giving a physical stimulus or chemical stimulus to the animal cell according to the variation in the tide-generating force.

### CROSS REFERENCE TO RELATED APPLICATION

The present application claims priority under 35 U.S.C. §119 of Japanese Patent Application No. 2015-206716, filed on Oct. 20, 2015, the disclosure of which is expressly incorporated by reference herein in its entirety.

### BACKGROUND OF THE INVENTION

Technical Field

The present invention relates to an animal cell regeneration speed controlling method and an animal cell regeneration speed controller, which make it possible to change a metabolism of the animal cell and to control the regeneration speed of the animal cell.

Related Art

In recent years, studies on regeneration methods intended, for example, to promote a regeneration speed are actively made in a regenerative medical field.

Systems, methods, devices and the like for promoting the growth of a regenerating tissue from a wound surface of a wound area in a biological tissue structure of a living human or animal body into the wound area in a predetermined direction are known as a specific technique for promoting a regeneration of a living tissue (see JP-A 2000-510712).

In the regeneration promoting methods according to JP-A 2000-510712, a growing direction of cell tissues is fixed by a mold or local drug concentration adjustment, resulting in promotion of regeneration. Therefore, such methods require a mold or local drug concentration adjustment for fixing the growing direction for each target tissue, and thus are disadvantageously apt to involve very complicated procedures and require a lot of time and efforts. Techniques that can promote the regeneration of cell tissues by an easier method are demanded.

### SUMMARY OF THE INVENTION

An object of the present invention is to provide an animal cell regeneration speed controlling method and an animal cell regeneration speed controller, that can change a metabolism of the animal cell and can easily control the regeneration speed of the animal cell.

The present inventor conducted intensive studies in view of the problems described above, found that a temporal change in tide-generating force indicated using, as an index, a relative value of gravity acceleration (relative gravity acceleration) based on the standard gravity acceleration as a reference unexpectedly acts on the regeneration of an animal cell, and that a physical stimulus or chemical stimulus is given to the animal cell according to the variation in tide-generating force, thereby allowing a change in metabolism of the animal cell, and finally achieved the present invention.

The present invention is as follows.

(1) An animal cell regeneration speed controlling method including:

grasping a tide-generating force; and

giving a physical stimulus or chemical stimulus to the animal cell according to the variation in the tide-generating force.

(2) The animal cell regeneration speed controlling method according to (1) above,

wherein relative gravity acceleration is used as an index of the tide-generating force.

(3) The animal cell regeneration speed controlling method according to (2) above,

wherein a stimulus is given to the animal cell in a time zone when the relative gravity acceleration changes from plus to minus based on the standard gravity acceleration as a reference.

(4) The animal cell regeneration speed controlling method according to (2) above,

wherein a stimulus is given to the animal cell in a time zone when the relative gravity acceleration changes from minus to plus based on the standard gravity acceleration as a reference.

(5) An animal cell regeneration speed controller comprising,

a means of grasping a tide-generating force (hereinafter, referred to also as “tide-generating force grasping means”), and

a stimulus controlling means of controlling a physical stimulus or chemical stimulus to be given to the animal cell according to the variation in the tide-generating force.

(6) The animal cell regeneration speed controller according to (5) above,

wherein the tide-generating force grasping means comprises a means of calculating relative gravity acceleration, and

wherein the stimulus controlling means comprises a means of controlling the physical stimulus or the chemical stimulus to be given to the animal cell according to the relative gravity acceleration.

(7) The animal cell regeneration speed controller according to (6) above,

wherein the animal cell regeneration speed controller gives a stimulus to the animal cell in a time zone when the relative gravity acceleration changes from plus to minus based on the standard gravity acceleration as a reference.

(8) The animal cell regeneration speed controller according to (6) above,

wherein the animal cell regeneration speed controller gives a stimulus to the animal cell in a time zone when the relative gravity acceleration changes from minus to plus based on the standard gravity acceleration as a reference.

In the present invention, the animal cell is, for example, a cell of mammals, birds, reptiles, amphibians, fish, or invertebrates such as poriferans, cnidarians, flatworms, mollusks, annelids, echinoderms and arthropod.

### Effect of the Invention

According to the animal cell regeneration speed controlling method of the present invention, the metabolism of animal cells can be easily changed according to the variation in tide-generating force to easily control the regeneration speed of the animal cells. That is to say, the regeneration speed of the animal cells can be promoted or suppressed according to an intended use. For example, when the regeneration of the animal cells is promoted, it is possible to improve the regeneration efficiency and shorten the regeneration period. Therefore, the regeneration cost can be reduced.

In the case where a relative gravity acceleration is used as an index of the tide-generating force, the tide-generating force may be easily grasped eliminating the necessity for special facilities and the metabolism of the animal cell can be effectively changed.

In the case where a physical stimulus or a chemical stimulus is given to the animal cell in a time zone when the relative gravity acceleration changes from plus to minus based on the standard gravity acceleration as a reference, the metabolism of the animal cell can be sufficiently changed. In particular, the regeneration speed of the animal cell can be promoted.

In the case where a physical stimulus or a chemical stimulus is given to the animal cell in a time zone when the relative gravity acceleration changes from minus to plus based on the standard gravity acceleration as a reference, the metabolism of the animal cell can be sufficiently changed. In particular, the regeneration speed of the animal cell can be suppressed and the method can also be applied, for example, to suppress cancer cells.

According to the animal cell regeneration speed controller of the present invention, the metabolism of animal cells can be easily changed according to the variation in tide-generating force to easily control the regeneration speed of the animal cells. That is to say, the regeneration speed of the animal cells can be promoted or suppressed according to an intended use. For example, when the regeneration of the animal cells is promoted, it is possible to improve the regeneration efficiency and shorten the regeneration period. Therefore, the regeneration cost can be reduced.

In the case where the animal cell regeneration speed controller of the present invention includes a means of calculating relative gravity acceleration as the tide-generating force grasping means, and the stimulus controlling means includes a means of controlling the physical stimulus or the chemical stimulus to be given to the animal cell according to the relative gravity acceleration, the tide-generating force may be easily grasped and the metabolism of the animal cell can be effectively changed.

In the case where the animal cell regeneration speed controller of the present invention gives a physical stimulus or a chemical stimulus to the animal cell in a time zone when the relative gravity acceleration changes from plus to minus based on the standard gravity acceleration as a reference, the metabolism of the animal cell can be sufficiently changed. In particular, the regeneration speed of the animal cell can be promoted.

In the case where the animal cell regeneration speed controller of the present invention gives a physical stimulus or a chemical stimulus to the animal cell in a time zone when the relative gravity acceleration changes from minus to plus based on the standard gravity acceleration as a reference, the metabolism of the animal cell can be sufficiently changed. In particular, the regeneration speed of the animal cell can be suppressed and the method can also be applied, for example, to suppress cancer cells.

### BRIEF DESCRIPTION OF THE DRAWINGS

FIG. 1 is a graph indicating a temporal change in relative gravity acceleration when the standard gravity acceleration (1G) is defined as a reference (zero point); and

FIG. 2 is a schematic diagram for explaining the cutting position of a planarian.

### DESCRIPTION OF THE EMBODIMENTS

### 1. Animal Cell Regeneration Speed Controlling Method

The animal cell regeneration speed controlling method of the present invention is characterized in including a step of grasping tide-generating force and a step of giving a physical stimulus or chemical stimulus to the animal cell according to the variation in the tide-generating force.

The tide-generating force may be indicated using, as an index, at least one of relative gravity acceleration (theoretical value), lunar calendar, weather data (atmospheric pressure and tidal level), and distance from the center of the earth to an implementation spot.

In the regeneration speed controlling method of the present invention, the relative gravity acceleration is especially preferably used as the index of the tide-generating force. Namely, it is preferable to grasp the relative gravity acceleration and to give a physical stimulus or chemical stimulus to the animal cell according to the relative gravity acceleration.

The relative gravity acceleration (RGA) means a relative value of gravity acceleration based on the standard gravity acceleration (1G=9.80665×10⁸ μGal) as a reference (zero point).

The relative gravity acceleration can be calculated by utilizing a commonly-publicized solid tidal force prediction program. Specifically, the relative gravity acceleration at a target spot and the temporal change thereof can be calculated by inputting information on the position of an implementation site (latitude and longitude), date (year, month and day) and time in the solid tidal force prediction program.

As the solid tidal force prediction program, the tide prediction system “GOTIC2” (http://www.miz.nao.ac.jp/staffs/nao99/) or the like can be used.

Examples of the physical stimulus to the animal cell include stimuli generated by cutting, piercing, vibration, pressure, tension, heat, light, acoustic wave, acceleration and electricity.

Examples of the chemical stimulus to the animal cell include stimuli generated by contact or administration of a chemical such as a drug.

Examples of the drug include substances having an action of promoting cell division such as substances having mitogen activity, plant lectins, plant hormones, Vitamin A derivatives, and molecular target agents which act on the cell division signal pathway; substances having the action of suppressing cell division such as anticancer agents, plant alkaloids and molecular target agents which act on the cell division signal pathway; and the like.

The physical stimulus and chemical stimulus to the animal cell may be combined with each other. The physical stimulus or chemical stimulus may be either a direct stimulus to an animal cell or an indirect stimulus, for example, to a living body surface having the cell.

In the case of giving a chemical stimulus using a drug, when the timing of administering the drug is adjusted according to the variation in tide-generating force without adjustment of the drug concentration, the differentiation/division speed of the animal cell can be controlled.

Specifically, when the drug effect of suppressing malignant cell growth is enhanced during culture of an animal cell, for example, in regenerative medicine, the suppressive effect can be enhanced by adjusting the timing of administration, not by increasing the drug concentration as conventional. As a result, the side effect of the drug suppressing malignant cell growth can be suppressed, and the growth efficiency of cells desired to increase finally can be improved. It is possible to reduce the amount of the drug, itself, to be used and to suppress the regeneration cost. Further, the number of complicated steps, for example, of adjusting the drug concentration can be reduced.

The physical stimulus or chemical stimulus to the animal cell is preferably given according to the variation in tide-generating force in the following specific time zones:

(1) a time zone when the relative gravity acceleration changes from plus to minus, based on the standard gravity acceleration as a reference; and

(2) a time zone when the relative gravity acceleration changes from minus to plus, based on the standard gravity acceleration as a reference.

The time zone (1) can be defined as a time zone which includes a time point when the relative gravity acceleration changes from plus to minus and during which the relative gravity acceleration is continuously descending, namely, the time zone until the relative gravity acceleration turns downward from the maximum value immediately before, passes through the zero point, and then turns upward.

The time zone (2) can be defined as a time zone which includes a time point when the relative gravity acceleration changes from minus to plus and during which the relative gravity acceleration is continuously ascending, namely, the time zone until the relative gravity acceleration turns upward from the minimum value immediately before, passes through the zero point, and then turns downward.

In the specific time zones, the timing of giving a stimulus to the animal cell is not particularly limited. In a case of the time zone when the relative gravity acceleration changes from plus to minus based on the standard gravity acceleration as a reference, the timing of stimulation is preferably within 4 hours, more preferably within 2 hours, even more preferably within 1 hour immediately after the relative gravity acceleration turns downward from the maximum value immediately before. On the other hand, in a case of the time zone when the relative gravity acceleration changes from minus to plus based on the standard gravity acceleration as a reference, the timing of stimulation is preferably within 4 hours, more preferably within 2 hours, even more preferably within 1 hour immediately after the relative gravity acceleration turns upward from the minimum value immediately before.

Conditions such as types of stimulus, amount thereof, number of times of stimulation and stimulation time are appropriately adjusted depending, for example, on the kind of animal cell to which the stimulus is to be given.

### 2. Animal Cell Regeneration Speed Controller

The animal cell regeneration speed controller of the present invention is intended for controlling the regeneration speed of an animal cell, and is characterized in including a tide-generating force grasping means, and a stimulus controlling means of controlling a physical stimulus or chemical stimulus to be given to the animal cell according to the variation in the tide-generating force.

Examples of the tide-generating force grasping means include a relative gravity acceleration calculation means in which the relative gravity acceleration is calculated, a means in which the tide-generating force is grasped from the lunar calendar, a means in which the tide-generating force is grasped from weather data such as atmospheric pressure and tidal level, a means in which a distance calculated from the center of the earth to an implementation spot to grasp the tide-generating force, and the like. Among these means, the relative gravity acceleration calculation means is preferred.

In the relative gravity acceleration calculation means, when information including the position of an implementation site (latitude and longitude), date (year, month and day) and time is input, the relative gravity acceleration at a target spot and the temporal change thereof are calculated. For the relative gravity acceleration, a commonly-publicized tidal force prediction program can be utilized, as described above.

In the stimulus controlling means, conditions for the physical stimulus or chemical stimulus to be imparted to the animal cell are controlled according to the variation in tide-generating force (for example, a data obtained by the tide-generating force grasping means, such as the calculated relative gravity acceleration).

In the present invention, the regeneration speed controller particularly preferably includes a relative gravity acceleration calculation means as the tide-generating force grasping means and a stimulus controlling means in which the physical stimulus or chemical stimulus to be imparted to the animal cell according to the calculated relative gravity acceleration is controlled.

For the purpose of controlling the stimulus according to the relative gravity acceleration, (1) a manner of giving a physical stimulus or chemical stimulus to an animal cell in a time zone when the relative gravity acceleration changes from plus to minus based on the standard gravity acceleration as a reference; or (2) a manner of giving a physical stimulus or chemical stimulus to an animal cell in a time zone when the relative gravity acceleration changes from minus to plus based on the standard gravity acceleration as a reference, may be conducted.

Conditions such as types of stimulus, amount thereof, number of times of stimulation and stimulation time are appropriately adjusted depending, for example, on the kind of animal cell to which the stimulus is to be given in the stimulus controlling means.

### 3. Industrial Applicability

According to the animal cell regeneration speed controlling method and the animal cell regeneration speed controller in the present invention, the metabolism of animal cells can be changed according to the variation in tide-generating force to easily control the regeneration speed of the animal cells (for example, promotion of regeneration). Therefore, the animal cell regeneration speed controlling method and the animal cell regeneration speed controller can be widely utilized in various fields associated with regeneration of animal cells (especially, regenerative medical field).

### EXAMPLES

Hereinafter, the present invention will be specifically described using Examples. In the following Examples 1 and 2, regeneration experiment was conducted using Planarians (Dugesia japonica) as one kind of flatworm.

### Example 1 (Animal Cell Regeneration Experiment (i))

Firstly, the tide prediction system “GOTIC2” (see, http://www.miz.nao.ac.jp/staffs/nao99/) was used to input the latitude and longitude of an implementation site (Kariya city, Aichi Prefecture) to calculate the time zone when the relative gravity acceleration (based on the standard gravity acceleration (1G) as a reference (zero point)), which served as an index of the tide-generating force, changed from plus to minus and the time zone when the relative gravity acceleration changed from minus to plus at an experiment place.

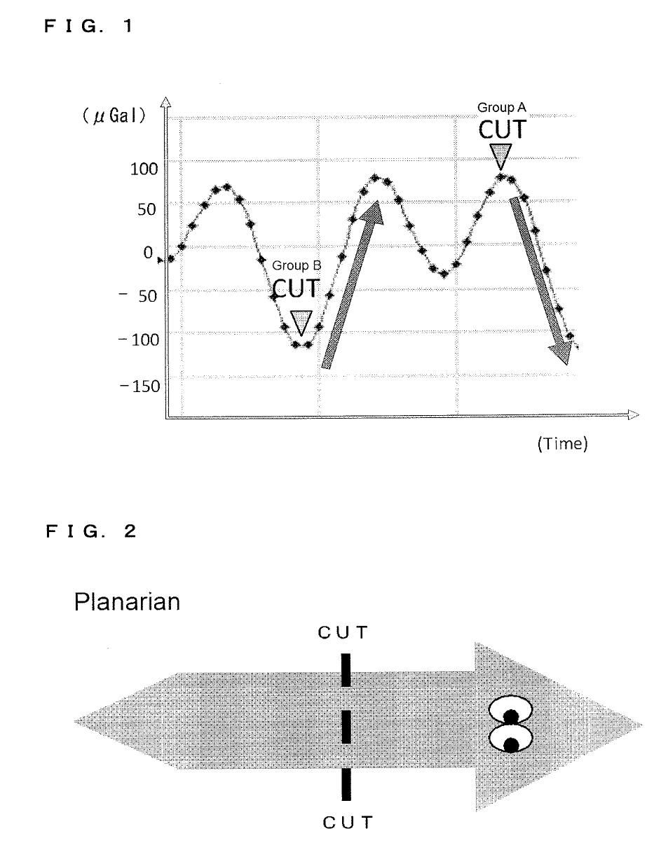

FIG. 1 shows a temporal change in the calculated relative value of gravity acceleration (relative gravity acceleration (μGal)). The arrow on the right side in FIG. 1 indicates a time zone when the relative gravity acceleration (RGA) changes from plus to minus, namely a time zone which includes a time point when the RGA changes from plus to minus and during which RGA is continuously descending. The arrow on the left side in FIG. 1 indicates a time zone when the RGA changes from minus to plus, namely a time zone which includes a time point when the RGA changes from minus to plus and during which the RGA is continuously ascending.

Then, planarians having an equivalent size were selected, and divided into Group A (n=9) and Group B (n=9). For the planarians belonging to Group A, the living bodies were cut near the center, immediately after the beginning of descending of the RGA (namely, immediately after the RGA turned downward from the maximum value immediately before (see “Group A-CUT” in FIG. 1)) in a time zone when the RGA changed from plus to minus (see FIG. 2).

For the planarians (n=9) belonging to Group B, the living bodies were cut near the center, immediately after the beginning of ascending of the RGA (namely, immediately after the RGA turned upward from the minimum value immediately before (see “Group B-CUT” in FIG. 1)) in a time zone when the RGA changed from minus to plus (see FIG. 2).

Thereafter, the planarians belonging to Groups A and B were respectively placed in petri dishes, so that the amount of water per planarian was 2.5 mL, in a thermostat bath set to a temperature of 13° C. The planarians were grown for 14 days (336 days) under a condition and in an environment such that water was changed every three days. The respective regeneration states of their heads in this period were observed over time with an optical microscope every 24 hours after cutting to evaluate the degree of regeneration after 14 days had elapsed based on the following three references (see Teresa Adel et al., Dev Genes Evol (2008) 218:89-103, as for the references for the degree of regeneration). The planarians ranked as “regeneration degree 2” or “regeneration degree 3” were determined to “have been regenerated,” and those ranked as “regeneration degree 1” were determined to “be unregenerated.” The number of planarians determined to “have been regenerated” was counted over time, and the result thereof is indicated in Table 1.

“Regeneration degree 1”: Eyes being developed cannot be identified.

“Regeneration degree 2”: Eyes being developed can be identified.

“Regeneration degree 3”: Eyes can be identified.

TABLE 1

| | Number of planarians determined to have | |
|---|---|---|
| Time elapsed after | been regenerated (cumulative) | |
| cutting (h) | Group A | Group B |
| 0 | 0 | 0 |
| 24 | 0 | 0 |
| 48 | 0 | 0 |
| 72 | 0 | 0 |
| 96 | 0 | 0 |
| 120 | 0 | 0 |
| 144 | 0 | 0 |
| 168 | 1 | 0 |
| 192 | 2 | 1 |
| 216 | 6 | 5 |
| 240 | 6 | 5 |
| 264 | 6 | 5 |
| 288 | 6 | 5 |
| 312 | 7 | 6 |
| 336 | 7 | 6 |

As is evident from Table 1, in Group A that was a group of planarians cut immediately after the beginning of descending of the RGA in the time zone when the RGA changed from plus to minus, one individual determined to “have been regenerated” appeared after 168 hours had elapsed since cutting; additional one individual appeared after 192 hours had elapsed; additional four individuals appeared after 216 hours had elapsed; and additional one individual appeared after 312 hours had elapsed. Among the total nine individuals, two individuals died and disappeared before regeneration.

On the other hand, in Group B that was a group of planarians cut immediately after the beginning of ascending of the RGA in the time zone when the RGA changed from minus to plus, one individual determined to “have been regenerated” appeared after 192 hours had elapsed since cutting; additional four individuals appeared after 216 hours had elapsed; and additional one individual appeared after 312 hours had elapsed. Among the total nine individuals, three individuals died and disappeared before regeneration.

This result could verify that the regeneration speed of the head (eyes) of Group A was increased more than that of Group B.

Consequently, it is considered that the degree of an influence of stimulus impartation (cutting treatment) on the metabolism of an animal cell varies depending on the timing of imparting a stimulus, and that the change has an influence also on the regeneration speed of the cell.

Therefore, when a physical stimulus by cutting treatment was given to an animal cell according to the variation in tide-generating force, the metabolism of the animal cell could be changed to control the regeneration speed. Especially, the regeneration speed could be promoted.

### Example 2 (Animal Cell Regeneration Experiment (ii))

Firstly, planarians having an equivalent size were selected, and divided into Group C (n=9) and Group D (n=9). For the planarians belonging to Group C, the living bodies were cut near the center at a timing in the same time zone as that for Group A in Example 1 (namely, immediately after the RGA turned downward from the maximum value immediately before) (see FIG. 2).

For the planarians (n=9) belonging to Group D, the living bodies were cut near the center at a timing in the same time zone as that for Group B in Example 1 (namely, immediately after the RGA turned upward from the minimum value immediately before) (see FIG. 2).

Thereafter, the planarians belonging to Groups C and D were respectively immersed for 24 hours in a solution of Azakenpaullone that is a drug suppressing normal differentiation at a concentration of 0.5 μM, then placed in petri dishes, so that the amount of water per planarian was 2.5 mL, in a thermostat bath set to a temperature of 13° C., and were grown for 15 days (360 days) under a condition and in an environment such that water was changed every three days. The respective regeneration states of their heads were observed over time with an optical microscope every 24 hours after cutting to evaluate the degree of regeneration after 15 days had elapsed in the same manner as in Example 1. At this time, the planarians were determined as to whether they “had been regenerated” or “were unregenerated” in the same manner as in Example 1. The number of planarians determined to “have been regenerated” was counted over time, and the result thereof is indicated in Table 2.

The treating method using Azakenpaullone was referred to a description of Teresa Adel et al., Dev Genes Evol 218(2008) 89-103.

TABLE 2

| | Number of planarians determined to have | |
|---|---|---|
| Time elapsed after | been regenerated (cumulative) | |
| cutting (h) | Group C | Group D |
| 0 | 0 | 0 |
| 24 | 0 | 0 |
| 48 | 0 | 0 |
| 72 | 0 | 0 |
| 96 | 0 | 0 |
| 120 | 0 | 0 |
| 144 | 0 | 0 |
| 168 | 0 | 0 |
| 192 | 0 | 0 |
| 216 | 1 | 0 |
| 240 | 2 | 6 |
| 264 | 2 | 6 |
| 288 | 3 | 6 |
| 312 | 4 | 6 |
| 336 | 4 | 6 |
| 360 | 5 | 6 |

As is evident from Table 2, in Group C that was a group of planarians cut immediately after the beginning of descending of the RGA in the time zone when the RGA changed from plus to minus, one individual determined to “have been regenerated” appeared after 216 hours had elapsed since cutting; additional one individual appeared after 240 hours had elapsed; additional one individual appeared after 288 hours had elapsed; additional one individual appeared after 312 hours had elapsed; and additional one individual appeared after 360 hours had elapsed. Among the total nine individuals, four individuals died and disappeared before regeneration.

On the other hand, in Group D that was a group of planarians cut immediately after the beginning of ascending of the RCA in the time zone when the RGA changed from minus to plus, six individuals determined to “have been regenerated” appeared after 240 hours had elapsed since cutting. Among the total nine individuals, three individuals died and disappeared before regeneration.

Group C was faster than Group D in terms of the time until the individual determined to “have been regenerated” appeared. According to the result, it is considered that the degree of an influence of stimulus impartation (cutting treatment and drug administration) on the metabolism of an animal cell varies depending on the timing of imparting a stimulus, and that the change has an influence also on the regeneration speed of the cell. The change in easiness of infiltration of the drug is considered to especially have an influence.

After 240 hours had elapsed since cutting, Group D showed a better regeneration efficiency than Group C. This is considered to have been due to an influence of secondary toxicity by high-concentration infiltration of the drug suppressing normal differentiation (Azakenpaullone).

Therefore, when a physical stimulus (cutting treatment) and chemical stimulus (drug administration) were given to an animal cell according to the variation in tide-generating force, the metabolism of the animal cell could be changed to control the regeneration speed.

It is noted that the foregoing examples have been provided merely for the purpose of explanation and are in no way to be construed as limiting of the present invention. While the present invention has been described with reference to exemplary embodiments, it is understood that the words which have been used herein are words of description and illustration, rather than words of limitation. Changes may be made, within the purview of the appended claims, as presently stated and as amended, without departing from the scope and spirit of the present invention in its aspects. Although the present invention has been described herein with reference to particular structures, materials and embodiments, the present invention is not intended to be limited to the particulars disclosed herein; rather, the present invention extends no all functionally equivalent structures, methods and uses, such as are within the scope of the appended claims.

The present invention is not limited to the above-described embodiments, and various variations and modifications may be possible without departing from the scope of the present invention.

What is claimed is:

1. An animal cell regeneration speed controlling method comprising:
grasping a tide-generating force; and
giving a physical stimulus or chemical stimulus to the animal cell according to the variation in the tide-generating force.

2. The animal cell regeneration speed controlling method according to claim 1,
wherein relative gravity acceleration is used as an index of the tide-generating force.

3. The animal cell regeneration speed controlling method according to claim 2,
wherein a stimulus is given to the animal cell in a time zone when the relative gravity acceleration changes from plus to minus based on the standard gravity acceleration as a reference.

4. The animal cell regeneration speed controlling method according to claim 2,
wherein a stimulus is given to the animal cell in a time zone when the relative gravity acceleration changes from minus to plus based on the standard gravity acceleration as a reference.

5. An animal cell regeneration speed controller comprising,
a means of grasping a tide-generating force, and
a stimulus controlling means of controlling a physical stimulus or chemical stimulus to be given to the animal cell according to the variation in the tide-generating force.

6. The animal cell regeneration speed controller according to claim 5,
wherein the tide-generating force grasping means comprises a means of calculating relative gravity acceleration, and
wherein the stimulus controlling means comprises a means of controlling the physical stimulus or the chemical stimulus to be given to the animal cell according to the relative gravity acceleration.

7. The animal cell regeneration speed controller according to claim 6,
wherein the stimulus controlling means gives a stimulus to the animal cell in a time zone when the relative gravity acceleration changes from plus to minus based on the standard gravity acceleration as a reference.

8. The animal cell regeneration speed controller according to claim 6,
wherein the stimulus controlling means gives a stimulus to the animal cell in a time zone when the relative gravity acceleration changes from minus to plus based on the standard gravity acceleration as a reference.
